(19) United States
(12) Patent Application Publication
(10) Pub. No.: US 2017/0112777 A1
(43) Pub. Date: Apr. 27, 2017

(54) PROTEIN-BASED PARTICLES FOR DRUG DELIVERY

(71) Applicant: Dana-Farber Cancer Institute, Inc., Boston, MA (US)

(72) Inventors: Zohreh AMOOZGAR, Brighton, MA (US); Michael Solomon GOLDBERG, Brookline, MA (US); Phil GRAYESKI, Raritan, NJ (US)

(73) Assignee: Dana-Farber Cancer Institute, Inc., Boston, MA (US)

(21) Appl. No.: 15/311,339

(22) PCT Filed: May 15, 2015
(86) PCT No.: PCT/US2015/031142

§ 371 Date: Nov. 15, 2016

Related U.S. Application Data
(60) Provisional application No. 61/994,157, filed on May 16, 2014.

(51) Int. Cl. A61K 9/51; A61K 31/704; A61K 31/4418; A61K 31/513; A61K 31/337; A61K 31/4439

(52) U.S. Cl. CPC A61K 9/5169; A61K 31/337; A61K 31/4439; A61K 31/4418; A61K 31/513; A61K 31/704

(57) ABSTRACT

In one aspect, a method for forming particles is provided. The method may allow biocompatible particles comprising an agent (e.g., pharmaceutically active agent) to be produced absent one or more purification step (e.g., removal of excess reagent). In certain embodiments, particles, produced as described herein, can be utilized in a pharmaceutical composition and/or administered to a subject without further purification. The lack of one or more purification step may simplify manufacturing and/or minimize or eliminate the loss of agent from the particle after formation. In some embodiments, the method comprises associating albumin with an agent and crosslinking to form particles, such that little or no cytotoxic molecules are produced and/or remain after particle formation. Cross-linked albumin particles formed via the methods described herein may serve as biocompatible carriers for a variety of agents.

### RELATED APPLICATION

The present application claims priority under 35 U.S.C. §119(e) to U.S. provisional patent application, U.S. Ser. No. 61/994,157, filed May 16, 2014, which is incorporated herein by reference.

### FIELD OF INVENTION

The present invention relates to protein-based particles for drug delivery, compositions thereof, methods for preparing such particles, and uses for the treatment of disease.

### BACKGROUND

Many applications in science and technology utilize particles as carriers for molecules. Certain applications require the particles to be biocompatible and have a relatively small diameter. For example, biocompatible nanoparticles are often used as delivery systems for pharmaceutically active agents as well as diagnostic agents. The use of nanoparticles allows the pharmaceutically active agent to be transported to and/or accumulate at the target site (i.e., the place of action), thereby minimizing undesirable side effects and lowering the required therapeutic dose. Moreover, biocompatible nanoparticles can be administered in vivo without inducing inflammation or other such adverse and undesired effects. Conventional methods for producing biocompatible nanoparticles are often complex, costly, or incompatible with certain classes of pharmaceutically active agents. Accordingly, improved compositions and methods for preparing such particles are needed.

### SUMMARY

The present invention provides methods for forming particles as well as the particles themselves, compositions, preparations, formulations, and kits useful for administration to a subject. The subject matter of the present invention involves, in some cases, interrelated products, alternative solutions to a particular problem, and/or a plurality of different uses of one or more systems and/or articles.

In one aspect, methods for preparing nanoparticles are provided. In some embodiments, the method may include providing a mixture of albumin and a pharmaceutically active agent, adding glutaraldehyde to the mixture of albumin and the pharmaceutically active agent, and crosslinking at least a portion of the albumin to form nanoparticles containing the pharmaceutically active agent and having an average cross-sectional dimension of less than or equal to about 200 nm. Following crosslinking less than about 10% of the initial amount of glutaraldehyde added remains in the mixture.

In another embodiment, the method includes providing a mixture of albumin and a pharmaceutically active agent, adding glutaraldehyde to the mixture of albumin and the pharmaceutically active agent, and crosslinking at least a portion of the albumin to form nanoparticles containing the pharmaceutically active agent. The method does not comprise removing excess glutaraldehyde.

The invention further provides methods of using the inventive particles. The particles may optionally be combined with a pharmaceutically acceptable excipient to form a pharmaceutical composition. The particles or a pharmaceutical composition thereof may be used to deliver a pharmaceutically active agent to a subject, including a human subject. Therefore, the inventive particles may be used to treat or prevent a pathological condition. The inventive particles or pharmaceutical compositions may also be included in conveniently packaged kits. The kits may include multiple dosage units, devices for administration of the particles, pharmaceutical excipients, and/or instructions for use. In some instances, the kits may be for use in the clinic by a medical professional.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### DEFINITIONS

The terms “administer,” “administering,” or “administration,” as used herein, refers to implanting, absorbing, ingesting, injecting, inhaling, or otherwise introducing an inventive particle, or a composition thereof, in or on a subject.

As used herein, the term “albumin” has its ordinary meaning in the art and may refer to a protein in the albumin family of globular proteins (e.g., serum albumin) that is naturally derived or chemically synthesized. Albumin preferably contains only natural amino acids, although non-natural amino acids (i.e., compounds that do not occur in nature but that can be incorporated into a polypeptide chain) and/or amino acid analogs as are known in the art may alternatively be employed. Also, one or more of the amino acids in the albumin may be modified, for example, by the addition of a chemical entity such as a carbohydrate group, a phosphate group, a farnesyl group, an isofarnesyl group, a fatty acid group, a linker for conjugation, functionalization, or other modification. None of the modifications should substantially interfere with the desired biological activity or property of the albumin or the ability to form particles, as described herein. The albumin may be from any species. In certain embodiments, the albumin is human or bovine albumin. In certain embodiments, the albumin is human albumin. The human albumin may have the following sequence: MKWVTFISLLFLFSSAY SRGVFRR DAH KSE VAHRFKDLGE ENFKALVLIA FAQYLQQCPF EDHVKLVNEV TEFAKTCVAD ESAENCDKSL HTLFGDKLCT VATLRETYGE MADCCAKQEP ERNECFLQHK DDNPNLPRLV RPEVDVMCTA FHDNEETFLK KYLYEIARRH PYFYAPELLF FAKRYKAAFT ECCQAADKAA CLLPKLDELR DEGKASSAKQ RLKCASLQKF GERAFKAWAV ARLSQRFPKA EFAEVSKLVT DLTKVHTECC HGDLLECADD RADLAKYICE NQDSISSKLK ECCEKPLLEK SHCIAEVEND EMPADLPSLA ADFVESKDVC KNYAEAKDVF LGMFLYEYAR RHPDYSVVLL LRLAKTYETT LEKCCAAADP HECYAKVFDE FKPLVEEPQN LIKQNCELFE QLGEYKFQNA LLVRYTKKVP QVSTPTLVEV SRNLGKVGSK CCKHPEAKRM PCAEDYLSVV LNQLCVLHEK TPVSDRVTKC CTESLVNRRP CFSALEVDET YVPKEFNAET FTFHADICTL SEKERQIKKQ TALVELVKHK PKATKEQLKA VMDDFAAFVE KCCKADDKET CFAEEGKKLV AASQAALGL (SEQ ID No. 1).

As used herein, the phrase “associated with” when used with respect to two or molecules (e.g., albumin and an agent) refers to a direct or indirect link between the molecules formed via a chemical and/or biological interaction. In some embodiments, the link is formed via a non-covalent bond (e.g., hydrogen bonding, van der Waals interactions, hydrophobic interactions, magnetic interactions, electrostatic interactions, pi stacking, dipole-dipole interactions, ligand-receptor interaction). In certain embodiments, the link is formed via a covalent bond (e.g., carbon-carbon bonds, disulfide bonds, ester bonds, amide bonds). In some embodiments, the link is formed via a biological binding event.

As used herein, the term “biocompatible” is intended to describe a material (e.g., particles, excipients) that is not toxic to cells. Particles are “biocompatible” if their addition to cells in vitro results in less than 20% cell death, and their administration in vivo does not induce inflammation or other such adverse effects.

As used herein, the term “mixture” has its ordinary meaning in the art and typically refers to a system that contains two or more chemical substances (e.g., proteins, peptides, compounds, elements) that are combined such that each substance retains its own chemical identity. The mixture may be homogenous (e.g., a solution) or heterogeneous (e.g., a suspension). In some embodiments, the mixture is homogenous (e.g., a solution). In certain embodiments, the mixture is heterogeneous (e.g., a suspension).

As used herein, the term “particle” refers to a small object, fragment, or piece of material and includes, without limitation, microparticles or nanoparticles. Particles may be composed of a single substance or multiple substances. In certain embodiments of the invention, the particle includes a crosslinked protein. In certain embodiments, the particles are substantially solid throughout. In some embodiments, a particle may not include or be a micelle, a liposome, or an emulsion. The term “nanoparticle” refers to a particle having a characteristic dimension (e.g., greatest dimension, average diameter) of less than about 1 micrometer and at least about 1 nanometer, where the characteristic dimension of the particle is the largest cross-sectional dimension of the particle. The term “microparticle” refers to a particle having a characteristic dimension of less than about 1 millimeter and at least about 1 micrometer, where the characteristic dimension of the particle is the smallest cross-sectional dimension of the particle.

The terms “composition” and “formulation” are used interchangeably.

As used herein, the terms “condition,” “disease,” and “disorder” are used interchangeably.

As used herein, the term “pharmaceutically active agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Pharmaceutically active agents include, without limitation, agents listed in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005. Preferably, though not necessarily, the pharmaceutically active agent is one that has already been deemed safe and effective for use in humans or animals by the appropriate governmental agency or regulatory body. For example, drugs approved for human use are listed by the FDA under 21 C.F.R. §§330.5, 331 through 361, and 440 through 460, incorporated herein by reference; drugs for veterinary use are listed by the FDA under 21 C.F.R. §§500 through 589, incorporated herein by reference. All listed drugs are considered acceptable for use in accordance with the present invention. In certain embodiments, the pharmaceutically active agent is a small molecule. Exemplary pharmaceutically active agents include, but are not limited to, anti-cancer agents, antibiotics, anti-viral agents, anesthetics, anti-coagulants, inhibitors of an enzyme, steroidal agents, steroidal or non-steroidal anti-inflammatory agents, antihistamines, immunosuppressant agents, antigens, vaccines, antibodies, decongestants, sedatives, opioids, pain-relieving agents, analgesics, anti-pyretics, hormones, prostaglandins, antihypertensives, receptor agonists, receptor antagonists, etc.

As used herein, the term “small molecule” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is at most about 1,000 g/mol, at most about 900 g/mol, at most about 800 g/mol, at most about 700 g/mol, at most about 600 g/mol, at most about 500 g/mol, at most about 400 g/mol, at most about 300 g/mol, at most about 200 g/mol, or at most about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and at most about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). In some embodiments, the small molecule may also be complexed with another molecule, such as a metal atom or ion.

As used herein, “SB-431542” refers to the drug developed by GlaxoSmithKline that is an inhibitor of the activin receptor-like kinase receptors, such as ALK5, ALK4, and ALK7. SB-431542 has the following structure:

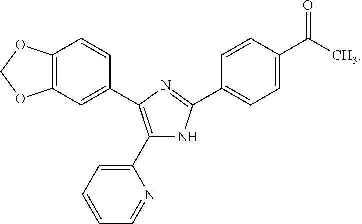 [SMILES: CC(=O)c1ccc(-c2nc(-c3ccc4c(c3)OCO4)c(-c3ccccn3)[nH]2)cc1]

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female at any stage of development. The animal may be a transgenic animal or genetically engineered animal. In certain embodiments, the subject is non-human animal. In certain embodiments, the animal is fish. A “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

As defined herein, the term “target tissue” refers to any biological tissue of a subject (including a group of cells, a body part, or an organ) or a part thereof, including blood and/or lymph vessels, which is the object to which a compound, particle, and/or composition of the invention is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

As used herein, the terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 provides confocal microscope images of albumin nanoparticles (top) and albumin nanoparticles modified with polyethylene glycol (i.e., PEGylated albumin nanoparticles) after incubation with J774A.1 macrophages for 1 hour.

FIGS. 2A-2C are (2A) a histogram of the cumulative release of pirfenidone from albumin nanoparticles containing a low dose of pirfenidone in serum simulated media at sink conditions, (2B) a histogram of the cumulative release of pirfenidone from albumin nanoparticles containing a high dose of pirfenidone in serum simulated media at sink conditions, and (2C) a graph of the cumulative release of pirfenidone from albumin nanoparticles containing a low dose of pirfenidone and albumin nanoparticles containing a high dose of pirfenidone in serum simulated media at sink conditions.

FIGS. 3A-3B are (3A) a histogram of percent cell viability of mouse breast cancer tumor model (4T1), mouse fibroblast (NIH3T3), human lung cancer cells (Calu6), and human ovarian cancer cells (SKOV3) in the presence of various concentrations of albumin nanoparticles, and (3B) a histogram of percent cell viability of 4T1, NIH3T3, Calu6, and SKOV3 cells in the presence of various concentrations of albumin nanoparticles containing pirfenidone.

FIGS. 4A-4C are graphs showing the viability of (4A) SKOV3 cells after exposure to free paclitaxel, free SB-431542, albumin nanoparticles containing paclitaxel, and albumin nanoparticles containing SB-431542 for 7 hours and 72 hours, (4B) Calu6 cells after exposure to free paclitaxel, free SB-431542, albumin nanoparticles containing paclitaxel, and albumin nanoparticles containing SB-431542 for 7 hours and 72 hours, and (4C) human foreskin fibroblasts (BR5) after exposure to a mixture of free paclitaxel and SB-431542 and albumin nanoparticles containing paclitaxel and SB-431542 for 7 hours and 72 hours.

FIGS. 5A-5C are (5A) a histogram of the cumulative release in serum simulated media at sink conditions of SB-431542 as a free drug and SB-431542 in a physical mixture with albumin in serum simulated media at sink conditions, (5B) a histogram of the cumulative release of paclitaxel as a free drug, paclitaxel in a physical mixture with albumin, and paclitaxel in albumin nanoparticles, and (5C) a histogram of the cumulative release in serum simulated media at sink conditions of SB-431542 and paclitaxel from albumin nanoparticles that contained both SB-431542 and paclitaxel.

FIGS. 6A-6B are transmission electron microscopy images of albumin nanoparticles.

FIG. 7 is a graph showing the tumor weight in mice after exposure to drug-free albumin particles; a combination of doxorubicin, 5-fluorouracil, and paclitaxel as a free drug mixture; and a combination of doxorubicin, 5-fluorouracil, and paclitaxel in an albumin nanoparticle.

FIG. 8 is a graph showing the weight of four week old mice, five week old mice, and six week old mice after exposure to a concentration of 4 mg/day or 40 mg/day of drug-free albumin for 16 days.

FIG. 9 is a histogram showing the population of immune cells in the spleen of mice after exposure to a concentration of 4 mg/day or40 mg/day of drug-free albumin for 16 days.

### DETAILED DESCRIPTION

Many applications in science and medicine utilize particles as carriers for pharmaceutically active agents. These applications often require the particles to be stable, biocompatible, and/or have a relatively small diameter. To produce such particles, many conventional methods utilize cytotoxic molecules, such as crosslinking agents, to form the particles and/or produce cytotoxic molecules as a result of particle formation and require one or more purification steps post particle formation to remove the cytotoxic molecules or unreacted crosslinking agent and render the particles biocompatible. However, in certain purification steps, at least a portion of the pharmaceutically active agent is removed along with the cytotoxic molecules. To circumvent this problem, other conventional methods utilize techniques that are complex and/or incompatible with certain classes of pharmaceutically active agents. A method has been discovered that does not require purification to form biocompatible particles comprising pharmaceutically active agents, and the method is compatible with a wide variety of pharmaceutically active agent.

In one aspect, a method for forming particles is provided. The method allows biocompatible particles comprising an agent (e.g., pharmaceutically active agent, diagnostic agents, contrast agents, prophylactic agents, nutrients) to be produced absent one or more purification steps (e.g., removal of excess reagent). In certain embodiments, particles, produced as described herein, are used in a pharmaceutical composition and/or administered to a subject without further purification. The lack of one or more purification steps simplifies manufacturing and minimizes or eliminates the loss of agent (e.g., small molecule) from the particles after formation. In some embodiments, the method comprises associating albumin with an agent and crosslinking the albumin to form particles, such that little or no cytotoxic molecules are produced and/or remain after particle formation. Crosslinked albumin particles formed via the methods described herein may improved release profiles compared to other particulate carriers and may serve as biocompatible carriers for a variety of agents.

In some embodiments, the method comprises associating albumin with the agent (e.g., pharmaceutically active agent) in a mixture (e.g., solution) and adding a dialdehyde (e.g., glutaraldehyde) to crosslink at least a portion of the albumin to form particles having a relatively small diameter (e.g., at least about 1 nm and less than 1,000 nm, less than or equal to 100 nm). For example, the method may include desolvating the albumin in a mixture to form albumin droplets in the mixture, adding the pharmaceutically active agent to the mixture, crosslinking the albumin to form particles, and harvesting the particles after crosslinking. In some embodiments, one or more parameters (e.g., concentration, crosslinking time, pH) may be controlled such that one or more purification step is not required to render the particles biocompatible prior to utilization. For instance, the concentration of glutaraldehyde and/or the crosslinking time may be controlled such that a relatively low amount of or no dialdehyde remains after particle formation. In one example, about 0.00008 w/v % and about 0.0005 w/v %, (e.g., between about 0.0001 w/v % and about 0.0003 w/v %) of dialdehyde may be added to an aqueous-based mixture (e.g., aqueous solution, aqueous-based solution comprising an alcohol) containing less than 10 w/v % albumin (e.g., less than or equal to about 5 w/v %) and allowed to crosslink for between about 4 and about 24 hours (e.g., between about 4 and about 12 hours) at 25° C. and at an alkaline pH (e.g., pH 9), such that substantially all the dialdehyde reacts with free amines of the albumin to form nanoparticles having a low coefficient of variation (e.g., less than or equal to about 20%, less than or equal to about 10%). In some such embodiments, the method does not include a step to remove excess dialdehyde (e.g., glutaraldehyde). The methods, described herein, may also have other advantageous properties including relatively high encapsulation efficiency (e.g., at least about 80%, at least about 90%) and loading (e.g., between about 2 wt. % and about 60 wt. %, at least about 10 wt. %).

It should be understood that one or more of the methods steps or the entire method, described herein, may be performed with or without agitation (e.g., stirring). For instance, the crosslinking step, associating albumin with an agent in a mixture, and/or the desolvating step may be performed with agitation.

As noted above, a crosslinking agent may be added to the mixture containing albumin and one or more agent (e.g., pharmaceutically active agent). In some embodiments, a relatively low concentration of crosslinking agent (e.g., dialdehyde) is used to form particles. By adding a relatively low amount of crosslinking agent, the amount of excess crosslinking agent remaining after particle formation is minimized. In certain embodiments, the concentration of crosslinking agent in the albumin mixture prior to crosslinking may be less than or equal to about 0.0005 w/v %, less than or equal to about 0.00045 w/v %, less than or equal to about 0.0004 w/v %, less than or equal to about 0.00035 w/v %, less than or equal to about 0.0003 w/v %, less than or equal to about 0.00025 w/v %, less than or equal to about 0.0002% w/v, less than or equal to about 0.00015 w/v %, less than or equal to about 0.0001% w/v. In some instances, the concentration of crosslinking agent in the albumin mixture prior to crosslinking may be between about 0.00008 w/v % and about 0.0005 w/v %, between about 0.00008 w/v % and about 0.0004 w/v %, between about 0.00008 w/v % and about 0.0003 w/v %, between about 0.0001 w/v % and about 0.0005 w/v %, between about 0.0001 w/v % and about 0.0004 w/v %, or between about 0.0001 w/v % and about 0.0003 w/v %. In some embodiments, the crosslinking agent is a dialdehyde. In certain embodiments, the crosslinking agent is glutaraldehyde. Without wishing to be bound by a particular theory, the high reactivity of dialdehydes with free amines, such as those in proteins, may help minimize the amount of excess crosslinking agent remaining after particle formation.

In some embodiments, the concentration of crosslinking agent in the albumin mixture prior to crosslinking is greater than 0.00008 w/v %. In some embodiments, the concentration of crosslinking agent in the albumin mixture prior to crosslinking is less than or equal to 0.0005 w/v %. In certain embodiments, when the concentration of crosslinking agent is greater than 0.005 w/v %, particles having a relatively small diameter (e.g., less than 1,000 nm, less than or equal to about 100 nm) cannot be formed using the methods described herein. In some such embodiments, a concentration of crosslinking agent greater than 0.005 w/v %, leads to the formation of gel matrices, aggregates, and/or particles having a relatively large diameter.

In some embodiments, a relatively small amount or no crosslinking agent remains in the mixture after the crosslinking step. For instance, in some embodiments, the concentration of crosslinking agent in the mixture after the crosslinking step may be less than or equal to about 0.0004 w/v %, less than or equal to about 0.0002 w/v %, less than or equal to about 0.0001 w/v %, less than or equal to about 0.00005 w/v %, less than or equal to about 0.000025 w/v %, less than or equal to about 0.00001% w/v, less than or equal to about 0.000005 w/v %, less than or equal to about 0.000001% w/v. In some instances, the concentration of crosslinking agent in the mixture after crosslinking is less than or equal to about 10⁻⁶ w/v % or less than or equal to about 10⁻⁷ w/v %. The concentration of crosslinking agent remaining in the mixture after crosslinking may be determined using high pressure liquid chromatography, liquid chromatography-mass spectrometry, nuclear magnetic resonance, or mass spectrometry. In certain embodiments, the crosslinking agent is undetectable after particle formation.

In some embodiments, the percentage of glutaraldehyde remaining in a mixture after the crosslinking step is less than to or equal to about 10%, less than or equal to about 5%, less than or equal to about 1%, less than or equal to about 0.5%, less than or equal to about 0.1%, less than or equal to about 0.05%, less than or equal to about 0.01%, less than or equal to about 0.005%, less than or equal to about 0.001%, less than or equal to about 0.0005%, or less than or equal to about 0.0001% of the initial amount of glutaraldehyde added to the mixture. In some instances, the percentage of glutaraldehyde remaining in a mixture after the crosslinking step is less than or equal to about less than or equal to about 0.0005%, or less than or equal to about 0.0001% of the initial amount of glutaraldehyde added to the mixture.

In some embodiments, the crosslinking agent is allowed to react with the albumin for a sufficient period of time to minimize or eliminate the amount of crosslinking agent remaining in the mixture (e.g., solution). For instance, in some embodiments, the total amount of time for the crosslinking step may be at least about 1 hour, at least about 4 hours, at least about 6 hours, at least about 8 hours, at least about 10 hours, at least about 12 hours, or at least about 16 hours. In some instances, the total amount of time for the crosslinking step may be between about 1 hour and about 24 hours, between about 1 hour and about 16 hours, between about 1 hour and about 12 hours, between about 4 hour and about 24 hours, between about 6 hour and about 24 hours, between about 8 hour and about 24 hours, between about 4 hour and about 16 hours, or between about 4 hour and about 12 hours. It should be understood that the above referenced times refer to crosslinking time in an aqueous based mixture (e.g., solution) at 25° C. and 1 atm. Other times may be used for other reaction conditions. For example, shorter or longer times may be required at higher or lower temperatures, respectively. The total amount of time for the crosslinking step may refer to the time from the addition of the crosslinking agent to the next method step (e.g., isolation of the particles, drying of the particles, functionalization of the particles).

In some embodiments, the agent (e.g., pharmaceutically active agent) may be incubated with the albumin for a period of time prior to crosslinking to allow at least a portion of the agent to associate with the albumin. For instance, in some embodiments, the pharmaceutically active agent may be incubated with the pharmaceutically active agent for at least about 0.1 hours, at least about 0.5 hours, at least about 1 hour, at least about 2 hours, at least about 4 hours, at least about 6 hours, at least about 8 hours, at least about 10 hours, at least about 12 hours, at least about 16 hours, or at least about 20 hours. In some embodiments, the pharmaceutically active agent may be incubated with the pharmaceutically active agent for between about 0.1 hours and about 24 hours, between about 0.1 hours and about 20 hours, between about 0.1 hours and about 16 hours, between about 0.1 hours and about 12 hours, between about 0.1 hours and about 8 hours, or between about 0.1 hours and about 4 hours depending on the agent.

Without being bound by theory, it is believed that a relatively large amount of the agent to be loaded into the particles may associate with the albumin leading to a relatively high encapsulation efficiency and/or loading. Certain plasma proteins, such as albumin, are biocompatible and capable of associating with a relatively large amount of a plethora of different molecules, including pharmaceutically active agents, via, e.g., non-covalent interactions. In some embodiments, the weight percentage of agent (e.g., pharmaceutically active agent) in the particles (i.e., loading) is at least about 0.5 wt. %, at least about 1 wt. %, at least about 2 wt. %, at least about 4 wt. %, at least about 6 wt. %, at least about 8 wt. %, at least about 10 wt. %, at least about 15 wt. %, at least about 20 wt. %, at least about 25 wt. %, at least about 30 wt. %, at least about 35 wt. %, at least about 40 wt. %, at least about 45 wt. %, or at least about 50 wt. %. In some instances, the loading is between about 0.5 wt. % and about 60 wt. %, between about 0.5 wt. % and about 50 wt. %, between about 0.5 wt. % and about 40 wt. %, between about 0.5 wt. % and about 30 wt. %, between about 1 wt. % and about 60 wt. %, between about 1 wt. % and about 50 wt. %, between about 1 wt. % and about 40 wt. %, between about 1 wt. % and about 30 wt. %, between about 2 wt. % and about 60 wt. %, between about 2 wt. % and about 50 wt. %, between about 2 wt. % and about 40 wt. %, or between about 2 wt. % and about 30 wt. %. The amount of agent loaded into the particles may be determined by extracting the agent from the dried particles using, e.g., organic solvents and measuring the quantity of the agent using high pressure liquid chromatography (i.e., HPLC), liquid chromatography-mass spectrometry, nuclear magnetic resonance, or mass spectrometry. Those of ordinary skill in the art would be knowledgeable of techniques to determine the quantity of an agent using the above-referenced techniques. For example, HPLC may be used to quantify the amount of an agent by, e.g., comparing the area under the curve of a HPLC chromatogram to a standard curve.

In some embodiments, albumin and one or more agents, to be loaded into the albumin particle, may associate via a chemical and/or biological interaction. In some embodiments, albumin and one or more agent may associate via a chemical interaction, such as a chemical bond. The chemical bond may be a covalent bond or non-covalent bond. In some cases, the chemical bond is a non-covalent bond such as a hydrogen bond, ionic bond, dative bond, and/or van der Waals interaction. In some embodiments, albumin and one or more agent may comprise functional groups capable of forming such bonds. For example, albumin may include at least one hydrogen atom capable of interacting with a pair of electrons on a hydrogen-bond acceptor of an agent to form the hydrogen bond. In some embodiments, albumin may include an electron-rich or electron-poor moiety, such that it may form an electrostatic interaction with an agent. In some embodiments, an association between albumin and one or more agent may occur via a biological binding event (i.e., between complementary pairs of biological molecules). For example, albumin may include a binding pocket for a particular agent.

In some embodiments, the encapsulation efficiency of the method is relatively high. For instance, in some embodiments, the encapsulation efficiency is at least about 80%, at least about 85%, at least about 90%, at least about 95%, at least about 97%, at least about 98%, at least about 99%, at least about 99.5%, or at least about 99.9%. As used herein, encapsulation efficiency refers to the ratio of the total amount of agent (e.g., pharmaceutically active agent) in the particles to the total amount of agent that was added during the particle formation process.

In some embodiments, the albumin is desolvated prior to associating the albumin with the agent (e.g., pharmaceutically active agent) and/or modifying the albumin. In general, albumin is water soluble and molecules (e.g. organic solvent molecules), preferably non-toxic, are used to desolvate the albumin. In certain embodiments, desolvated albumin may form smaller particles than solvated albumin. Those of ordinary skill in the art would be knowledgeable of suitable desolvating agents. Non-limiting examples of desolvating agents include alcohols and alcohol mixtures (e.g., methanol, ethanol, isopropanol, acetone), or salts (e.g., ammonium sulfate). In certain embodiments, the desolvating agent is an alcohol. In some cases, a halogenated solvent is not used as desolvating agent. In certain embodiments, a non-holgenated solvent is used.

In some embodiments, the method may comprise adding an additional component to the albumin. For instance, in some embodiments, the method may comprise attaching a chemical compound to albumin before or after association with the agent (e.g., pharmaceutically active agent), such that the chemical compound is present on the surface of the albumin particle. In some embodiments, an additional component (e.g., lipid, polymer, surfactant) may be associated with the albumin that is not attached to the surface of the particle. The albumin may be modified before or after particle formation. In certain embodiments, albumin is modified prior to crosslinking and/or association with agent. In general, any suitable chemical compound that can be attached to albumin. Non-limiting examples of chemical compounds include small molecules, polynucleotides, proteins, peptides, metals, polymers, oligomers, organometallic complexes, lipids, carbohydrates, etc. The chemical compound may modify any property of particle including surface charge, hydrophilicity, hydrophobicity, zeta potential, size, etc. In certain embodiments, the chemical compound is a polymer such as polyethylene glycol (PEG). In certain embodiments, the chemical compound is a targeting agent used to direct the albumin particles to a particular cell, collection of cells, tissue, or organ system and/or to promote endocytosis or phagocytosis of the particle. Any targeting agent known in the art of drug delivery may be used. Non-limiting examples of targeting agents include proteins, peptides, polynucleotides, small organic molecules, metals, metal complexes, carbohydrates, lipids, etc. In certain embodiments, the targeting agent is a protein or peptide. For example, a targeting agent may be an antibody (e.g., monoclonal antibody), an antibody fragment (e.g., Fab fragment), a protein receptor, a portion of a protein receptor, a peptide ligand (e.g., peptide hormone, signaling peptide, peptide ligand, etc.), a glycopeptide, or a glycoprotein. In certain embodiments, the targeting agent is a polynucleotide. For example, a targeting agent may be a DNA-based molecule, a RNA-based molecule, or an aptamer. In certain embodiments, the targeting agent is a carbohydrate. For example, a targeting agent may be a carbohydrate ligand a carbohydrate found on the surface of a cell. In certain embodiments, the targeting agent is small molecule. For example, a targeting agent may be an organic small molecule, an amino acid, or comprise a metal.

As described herein, the method may comprise providing a mixture of albumin and one or more agent (e.g., pharmaceutically active agent). In some embodiments, a relatively low concentration of albumin is used. Without being bound by theory, it is believed that certain concentrations of albumin prevents the formation of gel matrix and/or particle aggregates during crosslinking and allows relatively small particles (e.g., nanoparticles) to be formed. In some embodiments, the weight percentage of albumin in a mixture may be less than or equal to about 10% w/v, less than or equal to about 9% w/v, less than or equal to about 8% w/v, less than or equal to about 7% w/v, less than or equal to about 6% w/v, less than or equal to about 5% w/v, less than or equal to about 4% w/v, less than or equal to about 3% w/v, less than or equal to about 2% w/v, or less than or equal to about 1% w/v. In some instances, the weight percentage of albumin in a mixture may be between about 0.5% w/v and about 10% w/v, between about 0.5% w/v and about 9% w/v, between about 0.5% w/v and about 8% w/v, between about 0.5% w/v and about 6% w/v, between about 1% w/v and about 10% w/v, between about 1% w/v and about 8% w/v, or between about 1% w/v and about 6% w/v.

In some embodiments, after crosslinking the particles are dried. In general, any suitable drying method may be used. In certain embodiments, the particles may be flash frozen and lyophilized. Those of ordinary skill of the art would be knowledgeable of other suitable drying techniques, such as spray drying and heat drying under vacuum (e.g., rotary evaporation). Such suitable drying techniques are described in Abdelwahed et al., Freeze-drying of Nanoparticles: Formulation, Process and Storage Considerations, Adv Drug Deliv Rev. 58, 2006, at 1688-713 and Li et al., Nanoparticles by Spray Drying using Innovative New Technology: The Büchi Nano Spray Dryer B-90. Journal of Controlled Release, 147, 2010 at 304-10.

In general, the method may be performed under conditions suitable to limit the amount of cytotoxic molecules produced during or remaining after particle formation. In some embodiments, at least one method step (i.e., the entire method, the crosslinking reaction) may occur under relatively mild conditions, e.g., in an aqueous based mixture (e.g., solution) at 1 atm and room temperature. For instance, in some embodiments, crosslinking reaction may occur at a temperature between about 20° C. and about 30° C., between about 21° C. and about 30° C., between about 21° C. and about 25° C., or between about 25° C. and about 30° C. In some embodiments, the crosslinking reaction may occur at a temperature between about 20° C. and about 30° C. (e.g., 25° C.). In certain embodiments, any suitable temperature that does not lead to denaturation of the albumin may be used. In general, the temperature may be selected to prevent denaturation of the albumin.

In some embodiments, at least one method step (e.g., the entire method, the crosslinking step) is performed at an alkaline pH. For instance, in some embodiments, at least one method step is performed at a pH of at least about 8.0, at a pH of at least about 8.5, at a pH of at least about 9.0, at a pH of at least about 9.5, at a pH of at least about 10.0, or at a pH of at least about 10.5. In some instances, the pH may be between about 8.0 and about 11.0, between about 8.0 and about 10.5, between about 8.5 and about 10.5, or between about 8.5 and about 10.0. In certain embodiments, the crosslinking reaction is performed at an alkaline pH. In some such embodiments, the pH may enhance the reactivity of the crosslinking agent with the free amines. In some embodiments, the pH may be controlled by using a buffer (e.g., Tris buffer).

In some embodiments, the method may be performed with minimal use of non-aqueous solvents. For instance, in some embodiments, the amount of non-aqueous solvent in the mixture may be less than or equal to about 50%, less than or equal to about 40%, less than or equal to about 30%, less than or equal to about 20%, less than or equal to about 10%, less than or equal to about 5%, or less than or equal to about 1%. In some instances, the mixture may have 0% non-aqueous solvent. Exemplary solvents include organic solvents (e.g., chloroform, ethanol, isopropanol) and inorganic solvents. In certain embodiments, a halogenated solvent is not used. In embodiments in which the mixture contains a non-aqueous solvent, at least a portion of the solvent is evaporated prior to harvesting the particles.

In another aspect of the invention, particles formed via the methods described herein are provided. In some embodiments, the crosslinked albumin particles containing one or more agent (e.g., pharmaceutically active agent) may have a relatively small diameter. In certain embodiments, the particle is a nanoparticle. For instance, in some embodiments, the characteristic dimension (e.g., average diameter) of the particles is less than about 1,000 nm, less than or equal to about 800 nm, less than or equal to about 600 nm, less than or equal to about 500 nm, less than or equal to about 400 nm, less than or equal to about 300 nm, less than or equal to about 200 nm, less than or equal to about 100 nm, or less than or equal to about 50 nm. In some instances, the characteristic dimension (e.g., average diameter) of the particles is may be between about 10 nm and about 800 nm, between about 10 nm and about 600 nm, between about 10 nm and about 500 nm, between about 10 nm and about 400 nm, between about 10 nm and about 300 nm, between about 10 nm and about 200 nm, or between about 10 nm and about 100 nm. In some instances, the particles have a diameter less than or equal to 100 nm. In certain cases, the characteristic dimension of the particles is between about 10 nm and about 100 nm. As used herein, the diameter of a particle for a non-spherical particle is the diameter of a perfect mathematical sphere having the same volume as the non-spherical particle. In general, the particles are approximately spherical; however the particles are not necessarily spherical, but may assume other shapes as well. The measurements described herein typically represent the average particle size of a population. However, in certain embodiments, the measurements may represent the range of sizes found in a population, or the maximum or minimum size of particles found in the population.

In other embodiments, the particle is a microparticle. In certain embodiments, the particles may have an average diameter of less than 1 mm. For instance, in some embodiments, the average diameter of the particles is less than about 1,000 microns, less than or equal to about 500 microns, less than or equal to about 100 microns, less than or equal to about 50 microns, less than or equal to about 10 microns, or less than or equal to about 5 microns and greater than or equal to about 1 micron.

In some cases, the particles may have a narrow distribution in a characteristic dimension. For instance, in certain embodiments, the coefficient of variation of a characteristic dimension (e.g., diameter) of the particles may be less than or equal to about 20%, less than or equal to about 15%, less than or equal to about 10%, or less than or equal to about 5%.

In some embodiments, the particles may be biocompatible. For instance, in some embodiments, addition of the particles to cells in vitro results in less than or equal to about 20% cell death, less than or equal to about 15% cell death, less than or equal to about 12% cell death, less than or equal to about 10% cell death, less than or equal to about 8% cell death, less than or equal to about 5% cell death, less than or equal to about 3% cell death, less than or equal to about 2% cell death, or less than or equal to about 1% cell death and their administration in vivo does not induce inflammation or other such adverse effects.

In general, the particles are biodegradable. As used herein, “biodegradable” particles are those that, when introduced into cells, are broken down by the cellular machinery or by hydrolysis into components that the cells can either reuse or dispose of without significant toxic effects on the cells (i.e., fewer than about 20% of the cells are killed when the components are added to cells in vitro). The components preferably do not cause inflammation or other adverse effects in vivo. In certain embodiments, the chemical reactions relied upon to break down the biodegradable particles are catalyzed.

The particle may degrade over hours to days to weeks to months, thereby releasing the agent (e.g., pharmaceutically active agent) over an extended period of time. In certain embodiments, the half-life of the particle under physiological conditions is 1-72 hours (e.g., 1-48 hours, 1-24 hours). In certain embodiments, the half-life of the particle under physiological conditions is 1-7 days. In other embodiments, the half-life is from 2-4 weeks. In other embodiments, the half-life is approximately 1 month.

In some embodiments, the particle may be negatively charged. For instance, in some embodiments, the zeta potential of the particle may be between about −10 mV and about −30 mV, between about −5 mV and about −40 mV, between about −10 mV and about −40 mV, between about −15 mV and about −40 mV, between about −5 mV and about −35 mV, between about −5 mV and about −30 mV, between about −5 mV and about −25 mV, or between about −10 mV and about −25 mV.

As described herein, the albumin particle typically comprises one or more agents (e.g., pharmaceutically active agent). For instance, the particle may comprise two or more agents (e.g., two agents, three agents), such as two or more pharmaceutically active small molecules. The agent may be distributed throughout the particle, the agent may be found on the surface of the particle, and/or the agent may be found in the core of the particle. In general, the agent in the particle may be any suitable molecule. Non-limiting examples of classes of agent that the particle may comprise includes pharmaceutically active agents, prophylactic agents, nutrients, diagnostic agents, etc. In some embodiments, the pharmaceutically active agent is approved by the U.S. Food and Drug Administration for use in humans or for veterinary use. In certain embodiments, the agent is a prophylactic agent such as a vaccine. In certain embodiments, the agent is used to prevent pregnancy. In other embodiments, the agent is a nutritional supplement, a vitamin, or a mineral. In yet other embodiments, the agent is a diagnostic agent such as a contrast agent for imaging.

In some embodiments, the particle may optionally contain other components (e.g., chemical compound) in addition to the one or more agent. For example, the particle may comprise lipids, polymers, and/or surfactants. In some embodiments, the other components are not attached to the surface of the particle. In certain embodiments, the other components are included at low concentrations.

In some embodiments, after drying, the particles may form a powder. In some embodiments, the powder is a pillable powder. In some such embodiments, the powder may have a relatively high flowability as determined by angle of repose measurement or compressibility index. For instance, in some embodiments, the angle of repose is between about 25 degrees and about 45 degrees, between about 25 degrees and about 40 degrees, between about 25 degrees and about 35 degrees, or between about 25 degrees and about 30 degrees. In some instances, the angle of repose is between about between about 25 degrees and about 35 degrees (e.g., between about 25 degrees and about 30 degrees). In some embodiments, the angle of repose is less than or equal to about 45 degrees (e.g., less than or equal to about 40 degrees, less than or equal to about 35 degrees, less than or equal to about 30 degrees). In certain embodiments, the powder may have a relatively low compressibility index. For instance, in some embodiments, the compressibility index is less than or equal to about 25%, less than or equal to about 20%, less than or equal to about 15%, less than or equal to about 10%. In some cases, the compressibility index is less than or equal to about 15% (e.g., less than or equal to about 10%). The angle of repose and the compressibility index may be determined as specified in the U.S. Pharmacopoeia Chapter 1174 and European Pharmacopoeia Chapter 2.9.36.

Particles formed via the methods described herein may be particularly useful for administering an agent to a subject in need thereof. In some embodiments, the particles are used to deliver a pharmaceutically active agent. In some instances, the particles are used to deliver to deliver a prophylactic agent. In certain embodiments, the particles are used to deliver diagnostic agents, such as a contrast agent or labelled agent for imaging (e.g., CT, NMR, x-ray, ultrasound). The particles may be administered in any way known in the art of drug delivery, for example, orally, parenterally, intravenously, intramuscularly, subcutaneously, intradermally, transdermally, intrathecally, submucosally, sublingually, rectally, vaginally, etc.

In some embodiments, the albumin particles, formed as described herein, are particularly well-suited for the treatment of neoplastic disorders (e.g., cancer). In some neoplastic disorders, albumin is taken up by cells as a source of amino acids and/or energy. In some such embodiments, albumin particles may be more readily taken up by cancer cells than particles not having albumin on their surface. In certain embodiments, the relatively small average diameter of the particles (e.g., less than or equal to about 100 nm) may allow the particles to penetrate deeper into tumors than essentially identical particles that have a larger average diameter.

In some embodiments, the albumin particles are well-suited for the treatment of inflammatory disorders, such as rheumatoid arthritis, and other diseases that cause the porosity of arteries and veins to increase. In some such cases, the albumin particles may readily penetrate into the diseased tissue without adversely affecting healthy tissue. In some embodiments, the albumin particles may contain more than one agent (e.g., two small molecule pharmaceutically active agents, three small molecule pharmaceutically active agents).

In some such embodiments, the albumin particles are well suited for treating diseases (e.g., certain cancers) that require multiple pharmaceutically active agents.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue. Another exemplary malignant neoplasm is a hematopoietic cancer (e.g., leukemia).

In some embodiments, the particles may comprise one or more chemotherapeutic agents. Exemplary chemotherapeutic agents include, but are not limited to, anti-estrogens (e.g. tamoxifen, raloxifene, and megestrol), LHRH agonists (e.g., goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitro soureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca2+ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

Once the particles have been prepared, they may be combined with pharmaceutically acceptable excipients to form a pharmaceutical composition. As would be appreciated by one of skill in this art, the excipients may be chosen based on the route of administration as described below, the agent being delivered, and the time course of delivery of the agent.

Pharmaceutical compositions of the present invention and for use in accordance with the present invention may include a pharmaceutically acceptable excipient. As used herein, the term “pharmaceutically acceptable excipient” means a non-toxic, inert solid, semi-solid or liquid filler, diluent, encapsulating material or formulation auxiliary of any type. Some examples of materials which can serve as pharmaceutically acceptable excipients are sugars, such as lactose, glucose, and sucrose; starches, such as corn starch and potato starch; cellulose and its derivatives, such as sodium carboxymethyl cellulose, methylcellulose, hydroxypropylmethylcellulose, ethyl cellulose, and cellulose acetate; powdered tragacanth; malt; gelatin; talc; excipients, such as cocoa butter and suppository waxes; oils, such as peanut oil, cottonseed oil; safflower oil; sesame oil; olive oil; corn oil and soybean oil; glycols such as propylene glycol; esters, such as ethyl oleate and ethyl laurate; agar; detergents such as Tween 80; buffering agents such as magnesium hydroxide and aluminum hydroxide; alginic acid; pyrogen free water; isotonic saline; citric acid, acetate salts, Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., the particles), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants, such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, ethanol, U.S.P., and isotonic sodium chloride solution. In addition, sterile, fixed oils are conventionally employed as a solvent or suspending medium. For this purpose any bland fixed oil can be employed including synthetic mono or diglycerides. In addition, fatty acids such as oleic acid are used in the preparation of injectables.

The injectable formulations can be sterilized, for example, by filtration through a bacteria retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the inventive particles with suitable non irritating excipients or carriers such as cocoa butter, polyethylene glycol, or a suppository wax which are solid at ambient temperature but liquid at body temperature and therefore melt in the rectum or vaginal cavity and release the microparticles.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of an inventive pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also contemplated as being within the scope of this invention.

The ointments, pastes, creams, and gels may contain, in addition to the particles of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the particles of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the particles in a proper medium. Absorption enhancers can also be used to increase the flux of the compound across the skin. The rate can be controlled by either providing a rate controlling membrane or by dispersing the particles in a polymer matrix or gel.

The invention also provides kits for use in preparing or administering the inventive particles. A kit for forming particles may include the albumin and a dialdehyde as well as any solvents, solutions, buffer agents, acids, bases, salts, targeting agent, etc. needed in the particle formation process. Different kits may be available for different targeting agents. In certain embodiments, the kit includes materials or reagents for purifying, sizing, and/or characterizing the resulting particles. The kit may also include instructions on how to use the materials in the kit. The one or more agents (e.g., pharmaceutically active agent) to be encapsulated in the particle are typically provided by the user of the kit.

Kits are also provided for using or administering the inventive particles or pharmaceutical compositions thereof. The particles may be provided in convenient dosage units for administration to a subject. The kit may include multiple dosage units. For example, the kit may include 1-100 dosage units. In certain embodiments, the kit includes a week supply of dosage units, or a month supply of dosage units. In certain embodiments, the kit includes an even longer supply of dosage units. The kits may also include devices for administering the particles or a pharmaceutical composition thereof. Exemplary devices include syringes, spoons, measuring devices, etc. The kit may optionally include instructions for administering the inventive particles (e.g., prescribing information).

The following examples are intended to illustrate certain embodiments of the present invention, but do not exemplify the full scope of the invention.

### EXAMPLES

### Example 1

This example describes the formulation of biocompatible albumin nanoparticles. Several parameters including albumin concentration, glutaraldehyde concentration, and temperature were varied to determine the method conditions that would allow biocompatible albumin nanoparticle to be formed. A relatively low concentration of glutaraldehyde was needed to form nanoparticles that were biocompatible absent a purification step.

Particles were prepared by dissolving albumin in Tris Buffer (10 mM, pH 9) to form a 5 mL solution having a certain concentration of albumin and filtering the solution (0.45 μm -13 mm). Ethanol (4 mL, 100%) was added to the albumin solution, while stirring, to desolvate albumin into droplets. Then glutaraldehyde was added and the suspension was stirred overnight. The resulting suspension was flash frozen and lyophilized.

The parameters that were varied to determine the method conditions that would allow biocompatible albumin nanoparticles to be formed are shown in Table 1.

TABLE 1
Conditions for particle formation.

| | Albumin | Glutaraldehyde | glutaraldehyde | | |
|---|---|---|---|---|---|
| | conc. | conc. | added | Temp. | |
| Sample | (w/v) | (w/v) | (grams) | (° C.) | Outcome |
| 1 | 10% | 25% | 0.0005 | 25 | Gel formed |
| 2 | 10% | 25% | 0.000375 | 25 | Gel formed |
| 3 | 10% | 25% | 0.00025 | 25 | Gel formed |
| 4 | 10% | 25% | 0.000125 | 25 | Gel formed |
| 5 | 10% | 25% | 0.00005 | 25 | Gel formed |
| 6 | 10% | 25% | 0.0005 | 25 | Gel formed |
| 7 | 10% | 25% | 0.000375 | 25 | Gel formed |
| 8 | 10% | 25% | 0.00025 | 25 | Gel formed |
| 9 | 10% | 25% | 0.000125 | 25 | Gel formed |
| 10 | 10% | 25% | 0.00005 | 25 | Gel formed |
| 11 | 10% | 25% | 0.000025 | 25 | Microparticles formed |
| 12 | 10% | 25% | 0.0005 | 4 | Gel formed |
| 13 | 10% | 25% | 0.000375 | 4 | Gel formed |
| 14 | 10% | 25% | 0.00025 | 4 | Gel formed |
| 15 | 10% | 25% | 0.000125 | 4 | Microparticles formed |
| 16 | 10% | 25% | 0.00005 | 4 | Microparticles formed |
| 17 | 10% | 25% | 0.000025 | 4 | Microparticles formed |
| 18 | 5% | 25% | 0.0005 | 25 | Microparticles formed |
| 19 | 5% | 25% | 0.000375 | 25 | Microparticles formed |
| 20 | 5% | 25% | 0.00025 | 25 | Microparticles formed |
| 21 | 5% | 25% | 0.000125 | 25 | Microparticles formed |
| 22 | 5% | 25% | 0.00005 | 25 | Microparticles formed |
| 23 | 5% | 25% | 0.0005 | 4 | Microparticles formed |
| 24 | 5% | 25% | 0.000375 | 4 | Microparticles formed |
| 25 | 5% | 25% | 0.00025 | 4 | Microparticles formed |
| 26 | 5% | 25% | 0.000125 | 4 | Microparticles formed |
| 27 | 5% | 25% | 0.00005 | 4 | Microparticles formed |
| 28 | 5% | 25% | 0.000025 | 4 | Microparticles formed |
| 29 | 5% | 8% | 0.00016 | 25 | Large nanoparticles or |
| | | | | | microparticles formed; |
| | | | | | toxic |
| 30 | 5% | 8% | 0.00012 | 25 | Large nanoparticles or |
| | | | | | microparticles formed; |
| | | | | | toxic |
| 31 | 5% | 8% | 0.00008 | 25 | Large nanoparticles or |
| | | | | | microparticles formed |
| 32 | 5% | 8% | 0.00004 | 25 | Large nanoparticles or |
| | | | | | microparticles formed |
| 33 | 5% | 8% | 0.000016 | 25 | Nanoparticles formed |
| 34 | 5% | 8% | 0.000008 | 25 | Partial albumin solution |
| | | | | | remained |

In general, the higher concentration of albumin (i.e., 10 w/v %) led to gel formation at 25° C. Microparticles could be formed using 10 w/v % albumin by performing the crosslinking step at 4° C., which slowed down the reaction rate between the glutaraldehyde and the free amines of the albumin. However, nanoparticles were not formed using 10 w/v % albumin.

The lower concentration of albumin (i.e., 5 w/v %) allowed microparticles and nanoparticles to be formed. Nanoparticles were only formed when the relatively low concentration of glutaraldehyde was added to the albumin. When a relatively large amount of the glutaraldehyde was added to the 5 w/v % albumin solution, microparticles or a combination of microparticles and large nanoparticles formed. Samples 29-33, which formed nanoparticles, were tested for toxicity and the presence of glutaraldehyde in the solution after crosslinking. Toxicity was determined as described in Example 5. Samples 29 and 30 were found to have relatively low cell viability and a HPLC chromatogram of the solution after the crosslinking step showed a glutaraldehyde peak. The toxicity data for samples 29 and 30 can be seen in Table 2.

TABLE 2
Cell viability of samples 29 and 30.

| Particle | Sample 29 | Sample 30 |
|---|---|---|
| Concentration | Cell Viability | Cell Viability |
| (mg/mL) | (%) | (%) |
| 1 | 93.97 | 51.04 |
| 0.5 | 91.35 | 59.00 |
| 0.2 | 78.98 | 75.00 |
| 0.1 | 81.25 | 89.00 |
| 0.01 | 81.64 | 74.83 |

The HPLC chromatograms of the solution after the crosslinking step for samples 31 and 32 also showed a glutaraldehyde peak. Sample 33 was found to be biocompatible and the HPLC chromatograms did not show a glutaraldehyde peak. Sample condition 33 was used in Examples 2-4. Sample 34, which used the lowest amount (0.000008 grams) of glutaraldehyde, did not form particles.

### Example 2

This example described the formation of drug loaded albumin nanoparticles and methods of modifying the albumin. Albumin nanoparticles were able to encapsulate a variety of drugs as well as multiple drugs in a single particle with high loading and encapsulation efficiency.

Albumin particles, modified albumin particles, albumin particles comprising one or more drugs, and modified albumin particles comprising one or more drug were formed using the conditions in sample 33 in Example 1. Particles were characterized for the size, polydispersity, and surface charge using Zetasizer 90S (Malvern, Worcestershire, UK); the result of which are shown in Table 3. Particles comprising one or more drugs were also characterized for loading and encapsulation efficiency. Drug content was measured via HPLC analysis (Agilent, Santa Clara, Calif., USA) as described in Example 5. The loading and encapsulation efficiency of the drug loaded particles are shown in Table 4.

The particles had an average diameter of less than or equal to about 100 nm and had a negative surface charge. The negative charge was due to consumption of albumin primary amines during cross-linking. Loading ranged from about 2 wt. % to 30 wt. % and the encapsulation efficiency ranged from about 97% to about 120%.

TABLE 3
Size, polydispersity, and zeta potential
for various albumin particles.

| | Size | | Zeta |
|---|---|---|---|
| Particles | (nm) | Polydispersity | (mV) |
| Albumin | 63.9 ± 8.5 | 0.3 ± 0.06 | −18.3 ± 5.1 |
| AlbuminPEG | 52.9 ± 2.1 | 0.3 ± 0.1 | −16.5 ± 4.2 |
| PTX/AlbuminPEG | 35.4 ± 26.3 | 0.5 ± 0.2 | −20.3 ± 5.2 |
| SB-431542/AlbuminPEG | 44.9 ± 33.3 | 0.3 ± 0.1 | −16.4 ± 4.3 |
| PFN/Albumin-PEG | 101.4 ± 5.6 | 0.5 ± 0.2 | −15.2 ± 1.1 |
| PTX and SB-431542/ | 76.2 ± 5.9 | 0.3 ± 0.1 | −13.4 ± 2.3 |
| AlbuminPEG | | | |
| PTX and SB-431542 | 86 | — | — |
| and 5FU/AlbuminPEG | | | |

TABLE 4
Loading and encapsulation efficiency for various albumin particles.

| | | Encapsulation |
|---|---|---|
| | Loading | Efficiency |
| Particles | (wt. %) | (%) |
| PTX/AlbuminPEG | 3.2 ± 0.2 | 98.1 ± 9.1 |
| SB-431542/AlbuminPEG | 3.1 ± 0.4 | 97.3 ± 8.7 |
| PFN/Albumin-PEG | 24.9 ± 5.6 | 98.5 ± 7.2 |
| SB-431542 and PTX/ | | |
| AlbuminPEG | | |
| SB-431542 | 2.7 ± 0.2 | 99.3 ± 6.2 |
| PTX | 2.6 ± 0.1 | 100 ± 6.2 |
| PTX and SB-431542 and 5FU/ | | |
| AlbuminPEG | | |
| PTX | 2.56% | 107% |
| SB-431542 | 2.4% | 100.8% |
| 5FU | 28.5% | 119% |

### Example 3

This example describes the lack of cytotoxicity of the albumin nanoparticles described in Example 2. The albumin nanoparticles were biocompatible, and the PEGylated particles were able to evade macrophages.

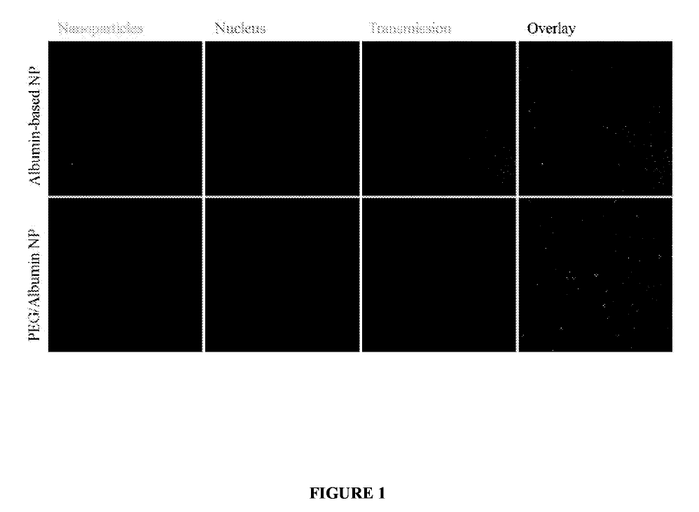

The ability of PEGylated albumin nanoparticles to resist rapid body clearance was evaluated in vitro as described in Example 5. As shown in FIG. 1, PEGylated particles were able to evade macrophages. J774A.1 macrophages were incubated with albumin or PEGylated albumin nanoparticles for 1 hour. PEGylated particles were not taken up by macrophages.

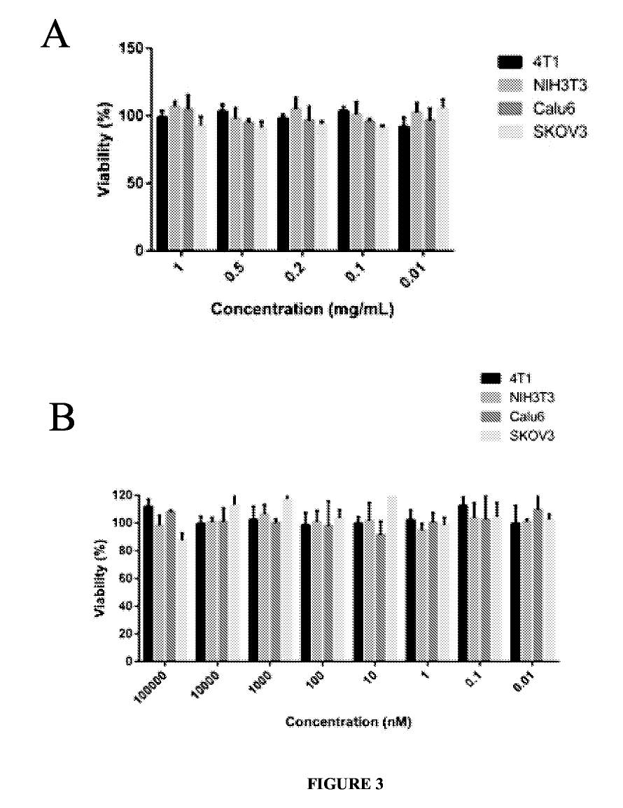

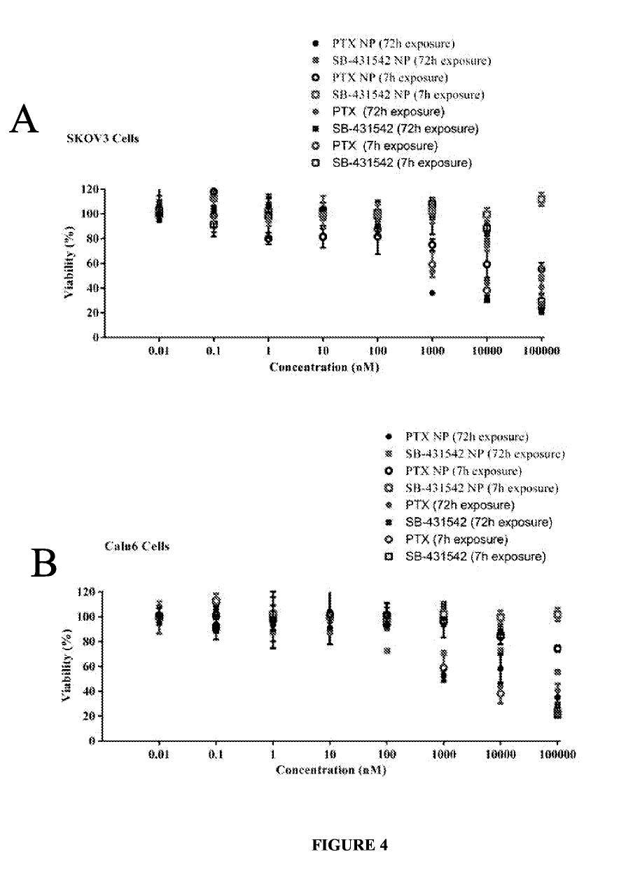

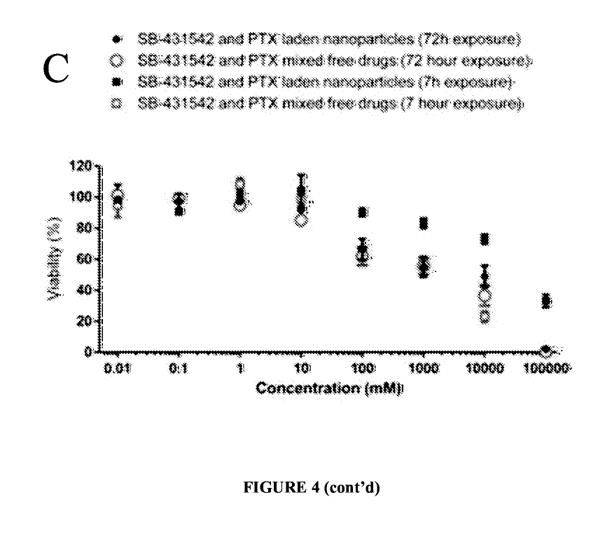

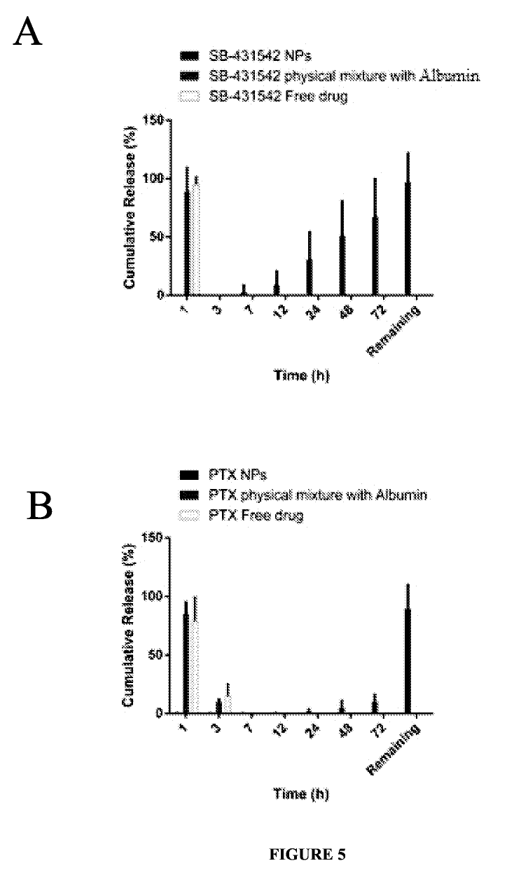

Cell viability upon exposure to drug-free nanoparticles and drug-laden nanoparticles was evaluated in vitro as described in Example 5 and the data is shown in FIGS. 3 and 4. Drug-free albumin-based nanoparticles were non-toxic to mouse breast cancer tumor model (4T1), mouse fibroblast (NIH3T3), human lung cancer cells (Calu6), and human ovarian cancer cells (SKOV3). This test highlighted the inertness of the drug delivery vehicle upon long exposure (72 hours) to a vast range of particle concentrations.

The cell viability of SKOV3 and Calu6 upon exposure to drug laden nanoparticles was evaluated in vitro as described in Example 5, and the data is shown in FIG. 4. Cell viability was evaluated after 7 hour and 72 hours. The cell viability of dual drug laden nanoparticles was tested on BR5 cells. The cytotoxicity of the particle to cancer cells was time and concentration dependent. Unlike free drug that was absorbed by cells, the nanoparticle cytotoxicity was dependent on the release kinetics of the drug from the nanoparticles. The release kinetics of the drugs from the particles is described in Example 4.

### Example 4

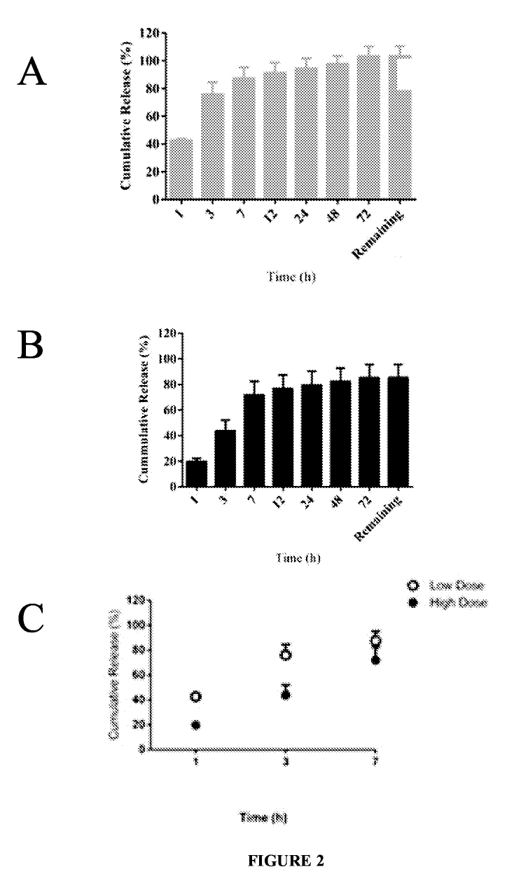

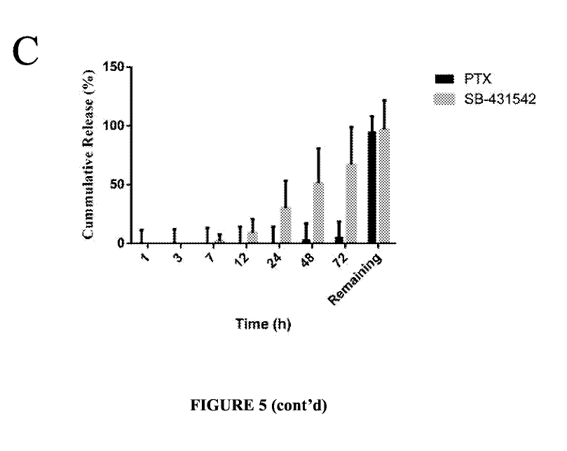

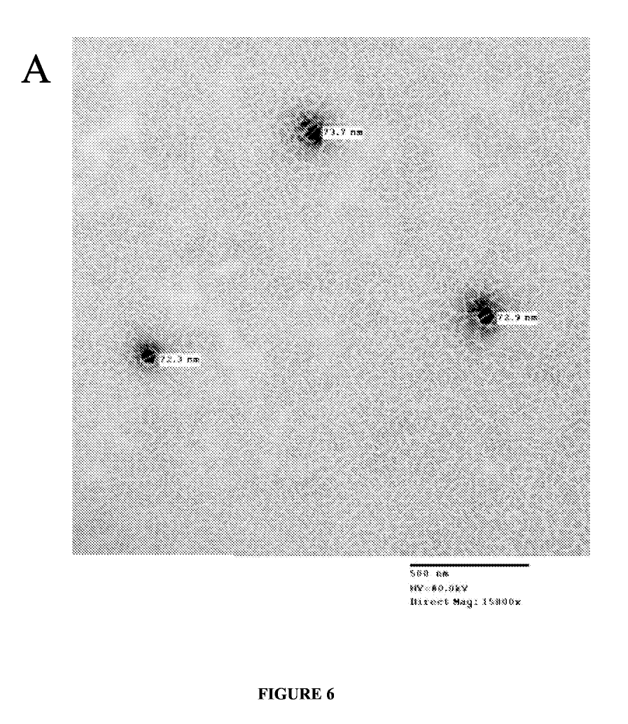

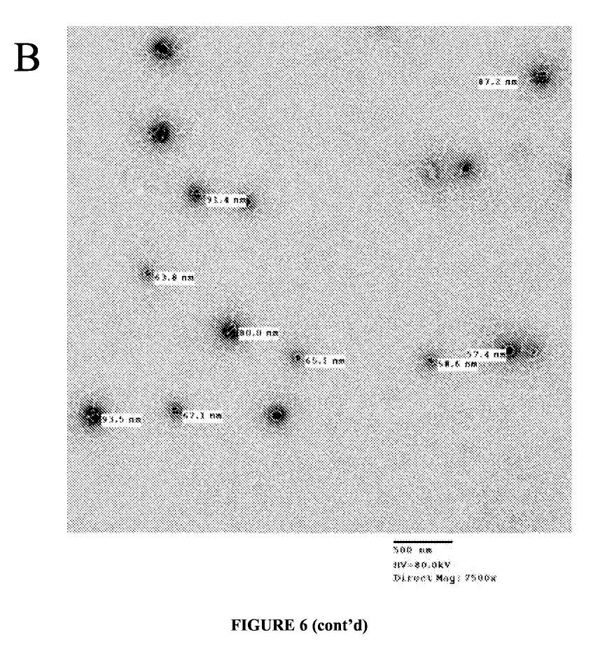

This example describes the release kinetics of drugs from the albumin nanoparticles described in Example 2. Release kinetics were performed in serum simulated media at sink condition and the amount of released drug was measured at predetermined intervals. FIGS. 2 and 5 shows the release kinetics of albumin particles comprising PFN, albumin particles comprising SB-431542, albumin particles comprising PTX, and albumin particles comprising SB-431542 and PTX.

PFN release started at the 1 hour time point and reached 70% in 7 hours, followed by a continuous release over 72 hours. SB-431542 release started at the 7 hour time point and reached about 70% in 72 hours following zero-order kinetics. Only 10% of PTX was released in the first 72 hours and the rest remained in nanoparticles. Dual-laden SB-431542 and PTX nanoparticles released the drugs independently and in similar manner to that of single-laden nanoparticles.

### Example 5

This example describes the methods used in Examples 1-4.

### Particle Formation

To form drug-free albumin particles, a 5% albumin solution was prepared by dissolving 2.5 g in 50 mL Tris Buffer (10 mM, pH 9) and filtered (0.45 μm -13 mm). Ethanol (4 mL, 100%) was added to 5 mL of the albumin solution, while stirring, to desolvate albumin into nanodroplets. Then 20 μL of 8% glutaraldehyde was added, and the suspension was stirred overnight. Nanoparticle suspension was flash frozen and lyophilized.

To form albumin particles modified with polyethylene glycol (PEG), 4-arm PEG (50 mg, pentaerythritol HCl salt, MW 5000) was added to 5 mL of 5% albumin solution, while stirring. Then, 4 mL 100% ethanol was added drop wise, and cross-linked with 20 μL of 8% glutaraldehyde overnight. Nanoparticle suspension was flash frozen and lyophilized.

To form albumin particles comprising paclitaxel, 5 mL 5% albumin solution and 50 mg 4-arm PEG under stirring, 10mg of paclitaxel (PTX) was added. The resulting suspension was stirred for ˜4 hours to pre-load PTX. After this time, 4 mL 100% ethanol was added drop wise, and nanodroplets were cross-linked via glutaraldehyde (20 μL of 8%). Nanoparticle suspension was flash frozen and lyophilized.

To form PEG-modified albumin particles comprising SB-431542, 5 mL 5% albumin solution and 50 mg 4-arm PEG under stirring, 10mg of SB-431542 solution in 1 mL 100% ethanol was added. Then, additional 3 mL 100% ethanol was added and was cross-linked via glutaraldehyde (20 μL of 8%) overnight. Nanoparticle suspension was flash frozen and lyophilized.

To form PEG-modified albumin particles comprising pirfenidone, 5 mL 5% albumin solution and 50 mg 4-arm PEG under stirring, 100mg of pirfenidone (PFN) was added. The resulting suspension was stirred for about 4 hours to pre-load PTX. After this time, 4 mL 100% ethanol was added drop wise, and nanodroplets were cross-linked via glutaraldehyde (20 μL of 8%). Nanoparticle suspension was flash frozen and lyophilized.

To form PEG modified albumin particles comprising paclitaxel and SB-431542, 5 mL 5% albumin solution and 50 mg 4-arm PEG under stirring, 10 mg of paclitaxel (PTX) was added. The resulting suspension was stirred for ˜4 hours to pre-load PTX. After this time, 10 mg of SB-431542 dissolved in 4 mL 100% ethanol was added drop wise, and nanodroplets were cross-linked via glutaraldehyde (20 μL of 8%). Nanoparticle suspension was flash frozen and lyophilized.

To form PEG-modified albumin particles comprising paclitaxel, 5-fluorouracil, and SB-431542, 5 mL 5% albumin solution and 50 mg 4-arm PEG under stirring, 10mg of paclitaxel (PTX) and 100mg 5-Fluorouracil (5-FU) was added. The resulting suspension was stirred for ˜4 hours to pre-load PTX. After this time, 10 mg of SB-431542 dissolved in 4.5 mL 100% ethanol was added drop-wise followed by 500 uL 100% DCM added drop-wise, and nanodroplets were cross-linked via glutaraldehyde (20 μL of 8%). Nanoparticle suspension was flash frozen and lyophilized.

### Drug-Loading Content Measurement

Loading of PTX, SB-431542, and PFN were measured after particle formation and freeze drying. Drugs were extracted using organic solvents and their quantity was measured according to AUC of their respective absorbance peaks (227 nm, 325 nm, and 311 nm respectively) compared to standard curve, by HPLC. For determination of PTX, SB-431542, PFN, and PTX/SB-431542 loading in nanoparticles, lyophilized nanoparticles were accurately weighed. For PTX and PFN, nanoparticles were dissolved in a 50:50 mixture of AcN and water. SB-431542 or PTX/SB-431542 nanoparticles were dissolved in 10:45:45 mixture of ethanol, AcN, and water, respectively. Solutions were filtered, and analyzed with high pressure liquid chromatography (HPLC) equipped with Ascentis C18-column (25 cm ×4.6 mm, particle size 5 μm). The mobile phase for PTX, PFN, SB-431542, and PTX/SB-431542 were 50:50, 70:30, 45:55, and 50:50 can respectively, with a flow rate of 1 mL/min, respectively. Peaks were detected using a UV detector at 227 nm (PTX), 311 nm (PFN), and 325 nm (SB-431542). PTX, PFN, and SB-431542 content in nanoparticles were calculated as a weight percentage of PTX and/or SB-431542 in nanoparticles. The encapsulation efficiency was determined by the ratio of drug in particles compared to initial added drug prior to particle formation and purification

### In Vitro Cell Viability Assay

Cytotoxicity of PTX, SB-431542, and PFN, nanoparticles loaded with PTX, SB-431542, and PTX/SB-431542 were evaluated using multiple cell lines including, but not limited to, Calu6, SKOV-3, and NIH3T3 cells via MTT assay. Cells were seeded at a density of 10,000 cells per well in a 96-well and incubated overnight in 200 μL of complete medium. The culture medium was then replaced with 198 μL of fresh medium, two free drug/DMSO solution or nanoparticle suspensions in the final concentration ranging from 0.001 to 100,000 nM. After 72 hours of incubation, the medium was replaced with 100 μL of fresh medium containing 13% MTT and incubated for 3.5 hours. Finally, 100 μL of the solubilization/stop solution comprising 20% SDS, 0.02% v/v acetic acid, and 50% v/v DMSO was added to each well, and the absorbance was read at 560 nm by a microplate reader. Cell viability was calculated by dividing the absorbance of treated cells by that of untreated cells after subtracting the absorbance of cell-free medium from each. Here, the untreated cells were those provided with no drug but handled equally otherwise, and the cell-free medium was the medium mixed with MTT solution and stop-solution without cells.

The MTT assay was also carried out limiting the cell exposure to nanoparticles or drug to 7 hours. Briefly, treatment containing media was removed after 7 hour exposure and replaced with fresh media. The cells were incubated for another 65 hours to determine the cell proliferation.

### Confocal Microscopy

Fluorescently labeled nanoparticles (plain albumin and PEGylated albumin) were prepared by replacing 25% of the albumin solution with fluoresceinamine-conjugated albumin. Sizes and zeta potentials of nanoparticles were measured prior to cell experiments. J774A.1 mouse macrophages (ATCC) were grown in DMEM. All media contained 10% fetal bovine serum (FBS) and 100 units/mL penicillin and 100 ug/mL streptomycin. J774A.1 cells were seeded at a density of 25,000 cells/cm² in a 35-mm dish with a glass window (MatTek). After overnight incubation, the medium was replaced with a 0.1 mg/mL of nanoparticle suspension in serum-free medium and incubated for 1 hour. Cells were then washed with 2 mL of serum-free medium twice to remove free or loosely-bound nanoparticles and observed using a Leica confocal microscope (Wetzlar, Germany). DRAQ-5 nuclear stain (1-2 μL) was added 2-3 minutes prior to imaging. Nanoparticles and cell nuclei were excited using a 488 nm and 633 nm laser respectively. Their emission signals were read from 500 to 600 nm and 650 to 750 nm and expressed in green and blue, respectively.

### Evaluation of Release Kinetics In Vitro

For release kinetics study, PTX, PFN, SB-431542 nanoparticles, free PTX, PFN or SB-431542, albumin physically mixed with PTX, PFN or SB-431542 (equivalent to 90 μg PTX or SB-431542 ; equivalent of 100 or 600 mg of PFN) were suspended in 1 mL of PBS (10 mM phosphate, pH 7.4) and transferred to a Tube-A-Lyzer® (Spectrum Laboratories Inc., Rancho Dominguez, Calif., USA) dialysis tube (MWCO: 20, 000). The tube was placed in 30 mL phosphate buffered saline (PBS, 10 mM phosphate, 137 mM NaCl, pH 7.4) containing 0.1% Tween 80, and incubated in a rotating shaker at 37° C. At pre-determined time points, the release medium was replaced with fresh release media, and the sampled medium was lyophilized. After the final collection at 72 hours, the remaining nanoparticles were collected from the dialysis tube and lyophilized. The release samples and the remaining nanoparticles were analyzed with HPLC.

### Example 6

This example describes the stability of albumin nanoparticles in dry powder form and in suspension. The dry powder was stable for several months in both ambient and reduced temperatures. The suspension of albumin nanoparticles in PBS was found to be stable after two freeze-thaw cycles.

Drug-free albumin nanoparticles were formed and dried to form a powder as described in Example 4. The stability of the drug-free nanoparticles at three different temperatures was assessed. Five hundred milligrams of the powder in a 20 mL glass screw cap vial was used to assess stability. The vials were stored either in a freezer having a temperature of about −20° C., in a refrigerator having a temperature between about 2° C. and about 8° C., and at ambient temperatures (i.e., between about 20° C. and about 25° C.). Every month, 3 samples from each vial were taken, suspended in PBS, and the particle size was measured. A statistically significant change (i.e., p-value≧0.05) in particle size was observed after 3 months at ambient temperatures, after 6 months at a temperature between about 2° C. and about 8° C., and after 12 months at a temperature of about 20° C.

To test the stability of suspended particles, the freeze/thaw stability test was performed. A suspension containing 5 mg/mL of albumin nanoparticles in PBS was prepared and cycled between about room temperature (i.e., 20±2° C.) and about −20±2° C. on 18-hour-freeze/6-hour-melt cycles for a total of 2 cycles. The size of the albumin nanoparticles remained unchanged.

Thus, albumin nanoparticles in dry powder form and in suspension were found to be very stable.

### Example 7

This example describes the use of albumin nanoparticles containing three FDA approved anti-cancer drugs, doxorubicin, fluorouracil, and paclitaxel, in a murine breast cancer model. The multi-drug albumin nanoparticles had a higher reduction in tumor weight than the free drug combination of doxorubicin, fluorouracil, and paclitaxel.

Doxorubicin, fluorouracil, and paclitaxel were encapsulated in albumin nanoparticles as described in Example 5, except doxorubicin—rather than SB-431542—was added along with paclitaxel and 5-FU. The multi-drug albumin nanoparticles had a size below 100 nm.

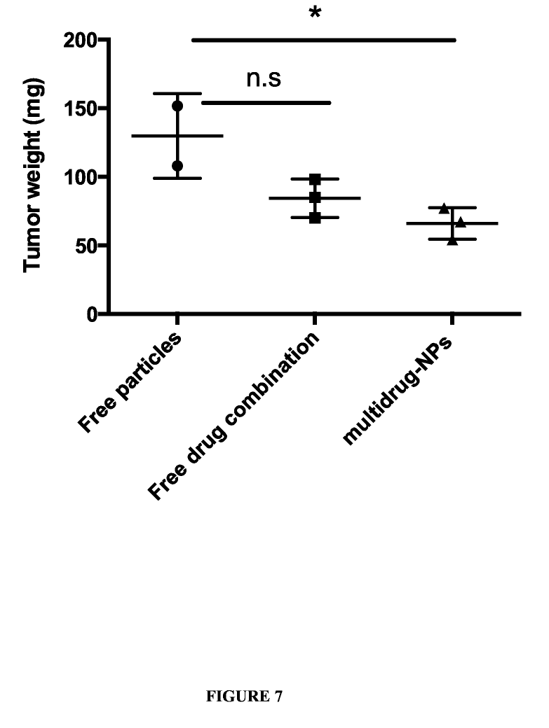

The ability of the albumin nanoparticles to reduce tumor weight in vivo was tested using the triple negative murine model of breast cancer (4T1). Fifty thousand 4T1 cells were inoculated subcutaneously into mice. Two days post tumor inoculation, the multi-drug albumin nanoparticles were injected intraperitoneally into the mice. As a control, the free drug combination was injected intraperitoneally into another group of mice two days post tumor inoculation. In addition, drug-free particles were injected two days post inoculation into a third group of mice as another control. The tumor weight is shown in FIG. 7. A significant reduction in tumor weight was observed for the multi-drug albumin nanoparticles.

### Example 8

This example describes the advantages of the sustained release capabilities afforded by the drug-loaded albumin nanoparticles. The use of albumin nanoparticles allowed a lethal dose of pirfenidone to be delivered to mice with minimal side effects.

Pirfenidone was dissolved in a 10% ethanol in PBS solution and administered intraperitoneally to healthy balb/C mice. The intraperitoneal pirfenidone dose was 200 mg/kg. This dose caused immediate neurological symptoms in the mice as well as death. However, the same dose was well-tolerated using albumin nanoparticles containing pirfenidone. The nanoparticles were injected twice daily for a total dose of 200 mg/kg per day for 16 days. This example highlights the ability of drug-loaded nanoparticles to slowly release drugs and lower their toxic side effects.

### Example 9

This example describes the biocompatibility, including immunogenicity of albumin nanoparticles, in vivo. The albumin nanoparticles were found to be biocompatible in vivo.

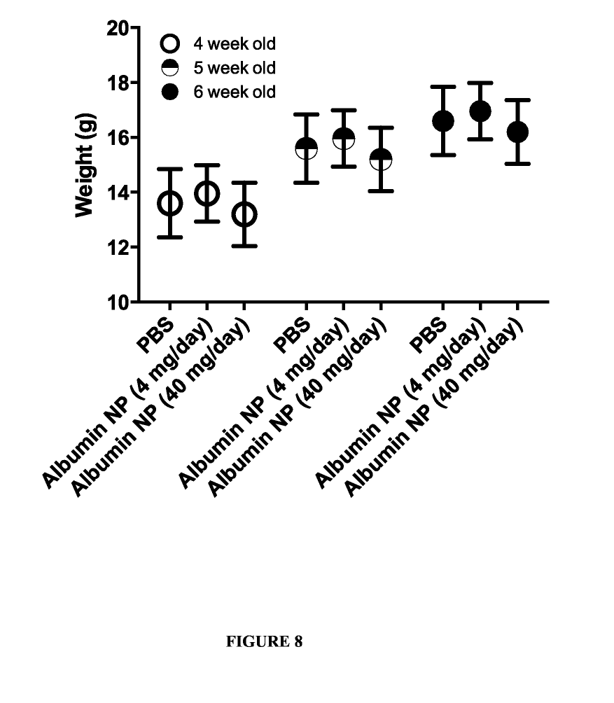

The biocompatibility of albumin nanoparticles was tested by injecting Balb/C mice twice daily with 2 mg of drug-free albumin nanoparticles (i.e., 4 mg/day), which represented a medium to high dose, for 16 days. Another group of Balb/C mice were injected twice daily with 20 mg of drug-free albumin nanoparticles (i.e., 40 mg/day), which represented an extremely high dose, for 16 days. Injection of PBS was used as a control. The health of mice, including body weight, was monitored throughout the 16 days. The body weight of the mice after 16 days based on the age of the mice is shown in FIG. 8. There was no relevant effect on the health and wellbeing of the mice after 16 days of drug-free albumin injections.

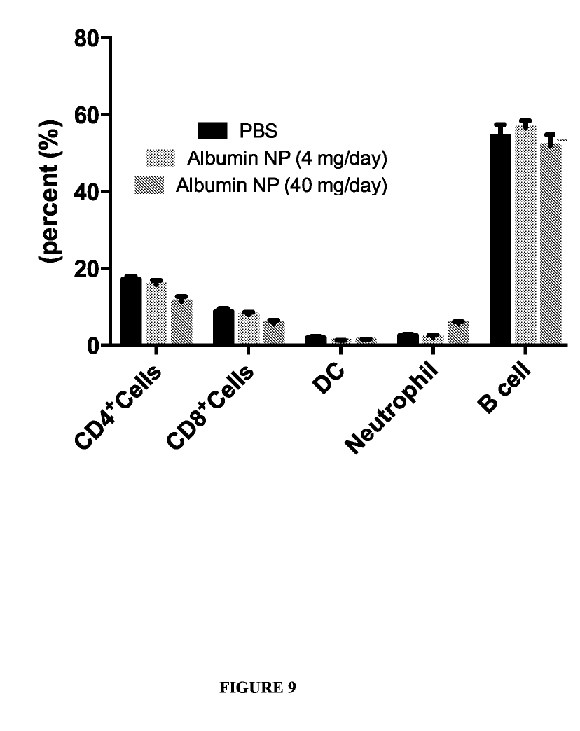

To access the immunogenicity of the albumin nanoparticles, the immune cell population in the spleen of the mice after 16 days was determined. FIG. 9 shows the immune cell distribution in the spleen of the mice. There was no statistical difference between the immune cell population of the mice receiving 4 mg/day or 40 mg/day drug-free albumin nanoparticles and the mice receiving PBS. Accordingly, the drug-free albumin particles were found to be biocompatible and non-immunogenic.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method for preparing nanoparticles comprising:
providing a mixture of albumin and a pharmaceutically active agent;
adding glutaraldehyde to the mixture of albumin and the pharmaceutically active agent; and
crosslinking at least a portion of the albumin to form nanoparticles containing the pharmaceutically active agent and having an average cross-sectional dimension of less than or equal to about 200 nm, wherein following crosslinking less than about 10% of the initial amount of glutaraldehyde added remains in the mixture.

2. A method for preparing nanoparticles, comprising:
providing a mixture of albumin and a pharmaceutically active agent;
adding glutaraldehyde to the mixture of albumin and the pharmaceutically active agent; and
crosslinking at least a portion of the albumin to form nanoparticles containing the pharmaceutically active agent, wherein the method does not comprise removing excess glutaraldehyde.

3. A method as in claim 1, further comprising the step of desolvating a portion of the albumin prior to the step of crosslinking.

4. A method as in claim 1, wherein the nanoparticles have an average cross-sectional dimension of less than or equal to about 100 nm.

5. A method as in claim 1, wherein the concentration of glutaraldehyde in the mixture prior to crosslinking is less than or equal to about 0.0001 w/v %.

6. A method as in claim 1, wherein the concentration of albumin in the mixture is less than or equal to about 5 w/v %.

7. A method as in claim 1, wherein the mixture comprises a second pharmaceutically active agent.

8. A method as in claim 1, wherein the mixture comprises a third pharmaceutically active agent.

9. A method as in claim 8, comprising crosslinking at least a portion of the albumin to form nanoparticles containing the pharmaceutically active agent, the second pharmaceutically active agent, and the third pharmaceutically active agent.

10. A method as in claim 1, wherein the encapsulation efficiency is greater than or equal to about 80%.

11. A method as in claim 1, wherein the encapsulation efficiency is greater than or equal to about 95%.

12. (canceled)

13. A method as in claim 1, wherein crosslinking comprises incubating glutaraldehyde with the albumin for at least about 1 hour.

14. A method as in claim 1, wherein crosslinking comprises incubating glutaraldehyde with the albumin for at between about 1 hour and about 12 hours.

15. A method as in claim 1, wherein crosslinking occurs at a temperature between about 20° C. and about 30° C.

16. A method as in claim 1, wherein following crosslinking less than about 0.1% of the initial amount of glutaraldehyde added remains in the mixture.

17. Particles formed by a method of claim 1.

18-28. (Canceled)

29. A composition comprising particles of claim 17.

30. A pharmaceutical composition, comprising:
a composition of claim 29; and
one or more pharmaceutically acceptable carriers, additives and/or diluents.

31. A kit for the treatment of a disease, comprising:
a composition of claim 29; and
instructions for use of the composition for treatment of the disease.

32. (canceled)

33. A method of treating a disease in a patient in need of treatment for the disease, comprising:
administering a composition of claim 29 to the patient.

34. (canceled)
